(19) United States
(12) Patent Application Publication
(10) Pub. No.: US 2015/0245982 A1
(43) Pub. Date: Sep. 3, 2015

(54) NAIL POLISH DRYING METHOD AND PRODUCT THEREFOR

(71) Applicant: 32 Degrees Limited, Nottingham (GB)

(72) Inventors: Angela Susan Eisenberg, London (GB); John Daniel Robert Pickering, Devon (GB)

(21) Appl. No.: 14/426,226

(22) PCT Filed: Sep. 5, 2012
(86) PCT No.: PCT/GB2012/000696

§ 371 Date: Mar. 5, 2015

(51) Int. Cl. A61K 8/04; A61K 8/33

(52) U.S. Cl. CPC A61K 8/046; A61K 8/33; A61Q 3/00

(57) ABSTRACT

The invention comprises a nail drying aerosol spray that comprises a combination of solvents having different rates of volatility. One of the solvents is dimethyl ether.

### FIELD OF THE INVENTION

This invention relates to a method of drying nail polish and to a product for use in carrying out the method.

### BACKGROUND TO THE INVENTION

Polish or enamel applied to finger and toenails can take a considerable time to dry. Obviously this inhibits the activity of those whose nails have just been painted. The general problem has been addressed in a number of ways. U.S. Pat. No. 6,238,651 and European Patent No. 0 869 764 describe fast drying nail polish compositions however, because a manicurist will typically apply a number of layers of differing enamels, each would have to be of the claimed composition. This limits choice in a field in which widespread choice is expected.

An alternative approach has been to apply drying accelerators to conventional polishes or enamels. Examples of such accelerators are described in Japanese Patent Application JP3081214, Swiss Patent Application CH665771, European Patent Application No. 0 774 173 and U.S. Pat. No. 5,804,169. All of these examples include different types of oils, most commonly silicone oils, and are thus not suitable for use when multiple layers of enamel need to be dried quickly. Further, oils tend to leave a greasy film when used.

It is an object of this invention which will go at least some way in addressing the drawbacks mentioned above; or which will at least provide a novel and useful alternative.

### SUMMARY OF THE INVENTION

In one aspect the invention provides a method of drying polish applied to nails, said method being characterized in that it includes drying subsequent applications using an aerosol-based drying product which does not inhibit the adhesion of subsequent applications of polish.

Preferably each application of polish is dried with said aerosol-based product before the next application.

In a second aspect the invention comprises an aerosol-based nail drying product for use in the method set forth above, said drying product including 5% to 90% by weight of dimethyl ether.

Preferably said drying product is substantially oil free.

In a third aspect the invention comprises an aerosol-based nail drying product for use in the method set forth above, said drying product including a plurality of solvents, each of said solvents having a different rate of volatility.

Preferably said drying product includes three different solvents.

Preferably one of said solvents comprises dimethyl ether.

Preferably said solvents further comprise isopentane and ethanol.

Preferably said drying product further includes one or more of a UV filter, a fragrance and a cooling active.

In a fourth aspect the invention comprises an aerosol-based nail drying product for use in the method set forth above, said drying product including propellant that also acts as a solvent.

Preferably said propellant and solvent comprises or includes dimethyl ether.

Many variations in the way the present invention can be performed will present themselves to those skilled in the art. The description which follows is intended as an illustration only of one means of performing the invention and the lack of description of variants or equivalents should not be regarded as limiting. Wherever possible, a description of a specific element should be deemed to include any and all equivalents thereof whether in existence now or in the future.

### DESCRIPTION OF WORKING EMBODIMENT

The present invention provides a method of drying polish or enamel applied to finger nails and toe nails. The method and composition herein described allows multiple layers of enamel to be applied in relatively quick succession without detracting from the integrity and performance of the final layer, and without leaving a greasy or slippery residue.

A characterizing feature of the invention is that each layer is dried in a very short time before application of the next layer. Conventionally only final layers have been dried.

The drying preparation which enables the method to be performed effectively is preferably in the form of an aerosol and is preferably free of oils and compounds based on silicone.

The drying preparation preferably includes dimethyl ether as the major active ingredient. The dimethyl ether may be present in an amount of between 5% and 90% by weight and, more preferably, between 30% and 50% by weight. A characteristic feature of the dimethyl ether is that it acts both as the propellant and also contributes to the drying function. Given that dimethyl ether is slightly polar, it is believed to have a degree of solvency in the nail enamel in contrast to conventional butane or propane propellants, and therefore interacts more effectively with the nail enamel.

In addition to the dimethyl ether the solvent blend may also include one or more additional volatile solvents such as, for example, isopentane and ethanol. The solvents may be chosen to have different rates of volatility and thus provide a graduated drying function. In this regard, dimethyl ether is highly volatile, isopentane very volatile and ethanol fairly volatile. It is believed that the combination of solvents of differing volatilities gives a drying profile that starts very fast, then slows, then slows further resulting in a tougher enamel through control of the evaporation profile.

The drying agent may further include one or more fragrances, UV filters and cooling agents. The fragrance is simply a matter of choice. Benzophenone-3 is a suitable UV filter and may be present in an amount of between 0% and 0.3%. Menthyl lactate provides a suitable cooling effect and may be present in an amount of 0% to 0.5%.

### EXAMPLE

A drying preparation as above described was prepared with the following composition:

Dimethyl ether: 40%

Isopentane: 34%

Ethanol 24.3%

Fragrance: 1%

Menthyl lactate 0.2%

Benzophenone-3: 0.1%

The resulting preparation exhibited good stability and dried each later of enamel in the order of ? minutes.

1-11. (canceled)

12. A method of drying polish applied in layers to nails, said method comprising:
drying individual layers of polish using an aerosol-based drying product which does not inhibit the adhesion of polish subsequently applied wherein said aerosol-based drying product contains 5% to 90% by weight of dimethyl ether.

13. A method as claimed in claim 12 wherein each application of polish is dried with said aerosol-based drying product before the next application.

14. An aerosol-based product for drying nail polish comprising 5% to 90% by weight of dimethyl ether.

15. An aerosol-based drying product as claimed in claim 14 comprising 30% to 50% by weight of dimethyl ether.

16. An aerosol-based drying product as claimed in claim 14 comprising a plurality of additional solvents, each of said solvents having a different rate of volatility.

17. An aerosol-based drying product as claimed in claim 16 wherein said additional solvents comprise isopentane and ethanol.

18. An aerosol-based drying product as claimed in claim 14 further including one or more of a UV filter, a fragrance and a cooling active.
